(19) United States
(12) Patent Application Publication
(10) Pub. No.: US 2022/0012475 A1
(43) Pub. Date: Jan. 13, 2022

(54) Application with Mood Recognition, Tracking, and Reactions

(71) Applicant: Mark H. Scott, San Diego, CA (US)

(72) Inventor: Mark H. Scott, San Diego, CA (US)

(21) Appl. No.: 17/153,392

(22) Filed: Jan. 20, 2021

Related U.S. Application Data
(60) Provisional application No. 62/963,668, filed on Jan. 21, 2020.

(51) Int. Cl. G06K 9/00; G16H 40/67; H04W 4/90

(52) U.S. Cl. CPC G06K 9/00302; H04W 4/90; G16H 40/67

(57) ABSTRACT

An application software program (app) which uses camera hardware on a device to process one or more images and/or video for the purposes of observing mood status and changes in mood status and intensity, displaying mood graphically and tracking it through time, and possibly taking action, suggesting action, communicating to others, or alerting others.

### RELATED APPLICATIONS

This application claims priority to U.S. provisional patent application No. 62/963,668, filed Jan. 21, 2020 and entitled “Application with Mood Recognition, Tracking, and Reactions,” which is incorporated herein in its entirety.

### FIELD OF THE INVENTION

This invention relates to the image and/or video capture, facial and partial facial recognition and software and data processing parts of a device, screen, computer, phone, smart phone, smart watch, wearable, VR interface, or tablet as they relate to determining, measuring, or diagnosing mood and changes in mood, depression, anger, happiness, as it relates to suggestions, actions, and communication for behavior modifications, medicine delivery, entertainment, interventions, and tracking.

### BACKGROUND OF THE INVENTION

A large number of persons in the US and around the world suffer from disorders like anxiety, depression, anger, seasonal affective disorder (SAD), and post-traumatic stress disorder (PTSD). Such persons achieve treatment through therapies, medicines, interventions, and behavior modifications.

There are many who are undiagnosed or misdiagnosed who may not be availing themselves of the appropriate treatments.

Many of those who are properly diagnosed may still be at risk, as their therapy is not fully effective due to medicine or therapy effectiveness or compliance.

The impact of mental health is staggering, with suicide rates rising from 11.6 per 100,000 in 2008 to 14.0 per 100,000 in 2017 in the US, according to the American Foundation for Suicide Prevention (AFSP), and from 10.6 per 100,000 in 1999.

The World Health Organization estimates that approximately 1 million people die each year from suicide, with a rate of 16 per 100,000 globally. Mental health disorders (particularly depression and substance abuse) are associated with more than 90% of all cases of suicide.

Reliable cross-country data about the number of attempted suicides each year is not available. In contrast to data on deaths by suicide, no country in the world reports to the WHO official statistics on attempted suicide.

According to figures from the Centers for Disease Control, in the US there are roughly 25 attempts for each suicide death; and for young adults aged 15-24 the ratio is much higher: there are approximately 100-200 suicide attempts for each suicide death.

Estimates of the economic costs for suicide are approximately $69 billion in 2015 according to the American Foundation for Suicide Prevention to as high as $93 billion in 2013, once underreporting is factored into the data, according to the American Association of Suicidology.

The costs related to depression are higher than those associated only with suicide. Annual costs related to major depressive disorder rose to $210.5 billion in 2010, according to a 2015 study published in the Journal of Clinical Psychiatry.

Recent studies have indicated that increased social media usage is correlated with increased depression and loneliness.

Many modern devices, phones, screens, computers, smart watches, wearables and tablets are outfitted with cameras capable of taking images, video, or other instantaneous or real-time imaging of facial expressions, partial facial expressions, and other facial recognition techniques that can be utilized to measure, diagnose, or determine mood, depression, anger, and happiness.

Many of these devices also possess the ability to gather or query the user for input of information about mood.

Many of these devices also possess the ability to gather other biometric data, such as pulse, temperature, sound level, tone of voice, color of skin, presence of moisture, such as sweat, tears, or even blood, and location which may be helpful in determining mood.

These devices also possess the ability to display data or provide notifications about medicine schedules or therapy schedules.

These devices may possess the ability to display data or provide notifications about tracking of mood levels related to device or app usage.

These devices may possess the ability to communicate mood levels to other persons, friends, family members, medical professionals, or institutions predetermined by the user for entertainment, therapy, or intervention purposes.

### SUMMARY OF THE DISCLOSURE

It is an object of the present invention to provide a mood measurement by observation of facial expression and changes in facial expression instantaneously or through time. Examples of observations may include, but are not limited to shape of mouth, diameter of nostrils, distance between eyebrows, redness of eyes presence or absence of tears and/or sweat, presence or absence of facial hair, signs of injury, and measurements between parts of the face relative to others, especially if compared to prior images or videos, and in particular when compared to other images or videos for which the user has assigned a mood value when previously solicited by the software and hardware, or when the user chose to assign a value at a time of his or her own choosing, unsolicited from the software.

Embodiments of the present invention are directed to an application or program capable of utilizing one or more cameras on a device, screen, computer, phone, smart phone, smart watch, wearable, VR interface, or tablet to capture one or more images or video of facial expression and changes in facial expression for the purposes of determining mood and changes in mood.

Embodiments of the present invention are directed to an application or program capable of utilizing one or more cameras on a device, screen, computer, phone, smart phone, smart watch, wearable, VR interface, or tablet to utilize existing facial recognition hardware and software within the device operating system to observe facial expression and changes in facial expression for the purposes of determining mood and changes in mood.

Embodiments of the present invention are directed to an application or program capable of utilizing one or more cameras on a device, screen, computer, phone, smart phone, smart watch, wearable, VR interface, or tablet to utilize proprietary facial recognition hardware and software to observe facial expression and changes in facial expression for the purposes of determining mood and changes in mood.

Embodiments of the present invention are directed to an application or program capable of utilizing one or more cameras on a device, screen, computer, phone, smart phone, smart watch, wearable, VR interface, or tablet to utilize third party facial recognition hardware and software to observe facial expression and changes in facial expression for the purposes of determining mood and changes in mood.

Embodiments of the present invention are directed to an application or program capable of utilizing one or more cameras on a device, screen, computer, phone, smart phone, smart watch, wearable, VR interface, or tablet and processing facial recognition hardware and software to observe facial expression and changes in facial expression together with querying the user intermittently about mood status (as compared to one or multiple “training sessions”) or accepting unsolicited user input about mood status, at times the user selects, for the purposes of determining mood and changes in mood.

Embodiments of the present invention are directed to an application or program capable of utilizing one or more cameras on a device, screen, computer, phone, smart phone, smart watch, wearable, VR interface, or tablet to observe facial expression and changes in facial expression for the purposes of tracking mood and changes in mood.

Embodiments of the present invention are directed to an application or program capable of utilizing one or more cameras on a device, screen, computer, phone, smart phone, smart watch, wearable, VR interface, or tablet observe facial expression and changes in facial expression for the purposes of plotting and displaying mood and changes in mood graphically.

Embodiments of the present invention are directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others wirelessly via local communication, such as Bluetooth.

Embodiments of the present invention are directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others wirelessly locally or more distantly, via communication tools such as wi-fi.

Embodiments of the present invention are directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others wirelessly locally, more distantly, or globally, via communication tools such as cellular or satellite wireless.

A preferred embodiment is directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others who have been selected by the user before the information is shared.

A preferred embodiment is directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others who have accepted the user's invitation to receive the information before the information is shared.

A preferred embodiment is directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others who are health care professionals (such as primary care physician, psychologist, psychiatrist, EMT, or similar) or first responder professionals (such as fire department personnel, police department personnel, 911 personnel or similar).

A preferred embodiment is directed to an application or program capable of utilizing one or more cameras on a device, screen, computer, phone, smart phone, smart watch, wearable, VR interface, or tablet to observe facial expression and changes in facial expression for the purposes of sharing mood and changes in mood with others who are health care institutions (such as primary care practice, psychologist or psychiatrist practice, ambulance service, or similar) or first responder institutions (such as fire department, police department, 911 call center, suicide hotline or similar).

A preferred embodiment is directed to an application or program capable of querying or receiving user input about mood simultaneous with facial expression and changes in facial expression imaging utilizing one or more cameras on a device, screen, computer, phone, smart phone, smart watch, wearable, VR interface, or tablet to improve mood determination algorithms.

A preferred embodiment is directed to an application or program capable of querying or receiving sounds simultaneous with facial expression and changes in facial expression imaging utilizing one or more microphones and cameras on a device, screen, computer, phone, smart phone, smart watch, wearable, VR interface, or tablet to improve mood determination algorithms.

A preferred embodiment is directed to an application or program capable of querying or receiving user input about mood simultaneous with facial expression and changes in facial expression imaging utilizing one or more cameras on a device, screen, computer, phone, smart phone, smart watch, wearable, VR interface, or tablet to determine mood and changes in mood.

A preferred embodiment is directed to an application or program capable of querying or receiving sounds simultaneous with facial expression and changes in facial expression imaging utilizing one or more microphones and cameras on a device, screen, computer, phone, smart phone, smart watch, wearable, VR interface, or tablet to determine mood and changes in mood.

A preferred embodiment is directed to an application or program capable of initiating actions directed at the user, preset by the user, and based upon mood determination, such as initiating a notification, closing or opening an app, updating a mood tracking graphic, reminding the user to take medication, suggesting the user take medication (within compliance), or causing a sound, vibration, image or some combination of these actions.

A preferred embodiment is directed to an application or program capable of initiating actions directed at others, preset by the user, and based upon mood determination, such as making a phone call, sending a text, initiating a notification on an app, updating a mood tracking graphic, or causing a sound, vibration, image or some combination of these actions.

A preferred embodiment is directed to an application or program capable of querying or receiving sounds simultaneous with facial expression and changes in facial expression imaging utilizing one or more microphones and cameras on a device, screen, computer, phone, smart phone, smart watch, wearable, VR interface, or tablet to assist in determining mood while the user is performing other functions on the phone (aka running in the background) for a period of time predetermined by the user or user's parents or guardians, including wirelessly (without manual user input), for example a period of a number of minutes, hours, or days.

A preferred embodiment is directed to an application or program capable of querying or receiving sounds simultaneous with facial expression and changes in facial expression imaging utilizing one or more microphones and cameras on a device, screen, computer, phone, smart phone, smart watch, wearable, VR interface, or tablet to assist in determining mood while the user is performing other functions on the phone (aka running in the background) continuously, if set to do so by the user or user's parents or guardians, including wirelessly (without manual user input), until the user or user's parents or guardians, including wirelessly (without manual user input), chooses to turn off this functionality.

A preferred embodiment is directed to an application or program capable of querying or receiving sounds simultaneous with facial expression and changes in facial expression imaging utilizing one or more microphones and cameras on a device, screen, computer, phone, smart phone, smart watch, wearable, VR interface, or tablet to assist in determining mood while the user is performing other functions on the phone (aka running in the background) for a period of time predetermined by the user or user's parents or guardians, including wirelessly (without manual user input), for example a period of a number of minutes, hours, or days, set to recur, for example on the anniversary of a past negative event each year, or during a stressful holiday period.

### BRIEF DESCRIPTION OF THE DRAWINGS

FIG. 1 is an illustration showing an exemplary embodiment of the present invention with facial recognition hardware and software evaluating user expression and changes in facial expression.

FIG. 2 is an illustration showing an exemplary embodiment of the present invention with facial recognition hardware and software evaluating user expression and changes in facial expression to track mood.

FIG. 3 is an illustration showing an exemplary embodiment of the present invention with facial recognition hardware and software evaluating user expression and changes in facial expression to track mood and represent mood tracking graphically.

FIG. 4 is an illustration showing an exemplary embodiment of the present invention with facial recognition hardware and software evaluating user expression and changes in facial expression to share mood information with others wirelessly.

FIG. 5A is an illustration showing an exemplary embodiment of the present invention with facial recognition hardware and software evaluating user expression and changes in facial expression to share mood information with others who have been pre-selected by the user and have accepted the user's invitation to share mood information wirelessly.

FIG. 5B is an illustration showing an exemplary embodiment of the present invention with facial recognition hardware and software evaluating user expression and changes in facial expression to share mood information with others who are health care professionals or first responder professionals wirelessly.

FIG. 5C is an illustration showing an exemplary embodiment of the present invention with facial recognition hardware and software evaluating user expression and changes in facial expression to share mood information with others who are health care institutions or first responder institutions wirelessly.

FIG. 6 is an illustration showing an exemplary embodiment of the present invention with an application or program capable of querying or receiving user input about mood simultaneous with facial recognition hardware and software evaluating user expression and changes in facial expression to improve mood determination algorithms.

FIG. 7 is an illustration showing an exemplary embodiment of the present invention with an application or program capable of querying or receiving user input about mood simultaneous with facial recognition hardware and software evaluating user expression and changes in facial expression to determine mood.

FIG. 8 is an illustration showing an exemplary embodiment of the present invention with an application or program capable of querying or receiving sounds simultaneous with facial recognition hardware and software evaluating user expression and changes in facial expression to determine mood.

FIG. 9A is an illustration showing an exemplary embodiment of the present invention with an application or program capable of initiating actions directed at the user, preset by the user, and based upon mood determination.

FIG. 9B is an illustration showing an exemplary embodiment of the present invention with an application or program capable of initiating actions directed at others, preset by the user, and based upon mood determination.

FIG. 10 is an illustration showing an exemplary embodiment of the present invention with an application or program capable of querying or receiving information inputs from wearables or biological sensors simultaneous with facial recognition hardware and software evaluating user expression and changes in facial expression to determine mood.

FIG. 11 is an illustration showing an exemplary embodiment of the present invention with an application or program capable of prompting the user to manually interact with a medicine delivery device, such as an infusion set, to deliver or adjust a continuous (basal), intermittent, or one-time (bolus) dose of medicine.

FIG. 12 is an illustration showing an exemplary embodiment of the present invention with an application or program capable of interacting with a medicine delivery device, such as a medicine pump and infusion set, to deliver or adjust a continuous (basal), intermittent, or one-time (bolus) dose of medicine.

FIG. 13 is an illustration showing an exemplary embodiment of the present invention with an application or program capable of interacting with a medicine delivery device, such as a patch pump infusion set in a feedback loop, to automatically (without manual user input) deliver or adjust a continuous (basal), intermittent, or one-time (bolus) dose of medicine.

FIG. 14 is an illustration showing an exemplary embodiment of the present invention with an application or program capable of interacting with a medicine delivery device, such as an infusion set, to allow others, such as family members, first responders, or healthcare professionals with prior consent of user or user's parents or guardians to wirelessly (without manual user input) deliver or adjust a continuous (basal), intermittent, or one-time (bolus) dose of medicine.

FIG. 15 is an illustration showing an exemplary embodiment of the present invention with facial recognition hardware and software for evaluating user expression and changes in facial expression to track mood and capable of measuring biometric data including, but not limited to: distance between eyebrows, angle of eyebrows, distance from corners of mouth to bottom of eyes, etc. This list is illustrative, and not exhaustive.

FIG. 16 is an illustration showing an exemplary embodiment of the present invention flowchart with facial recognition and measurement hardware and software for evaluating mood, mood velocity, and mood acceleration, and taking actions based upon the values measured and calculated from that data, and displaying if the user is in contact with others on a tracking app.

### DETAILED DESCRIPTION OF THE EXEMPLARY EMBODIMENTS

The embodiments and examples shown here are to provide enough information to fully understand the invention. One skilled in the art will understand how minor changes or deviations can be made and still be within the scope of the invention. The following description of exemplary embodiments of the invention is not intended to limit the scope of the invention to these exemplary embodiments, but rather to enable any person skilled in the art to make and use the invention. To assist in a clear and unambiguous understanding of the disclosure, the following definitions are used:

### Definitions

A VR Interface is glasses, goggles, or a headset that may also utilize gloves (or other manual or digital interaction), speakers or headphones, or other integral auditory interaction. It may also include body position, location, leg or foot movement, head position, or other display or feedback information. The VR interface is usually used to provide a more immersive user experience than keyboard, screen, mouse or other pointing device, joystick, and other legacy computing, gaming, or communication interfaces that do not block out environmental information inputs to the user.
Mood recognition is the ability to discern user positive or negative emotions, such as happiness, anger, sadness, or indifference, as well as intensity, and rate of change of intensity.
An app is an application program, or software that is generally not hardware or firmware on a device. An app may be preinstalled on a device at time of purchase, or may be added later. An app may also later be incorporated into device firmware.
A bolus is generally a single dose of medicine, typically a larger dose administered at once and often coincident with an external event, such as a specific dose of insulin at mealtime. The purpose is to provide additional medicine to help the body address the external event, such as carbohydrate intake of the meal. A bolus may also be given if there are other foreseen or unforeseen events affecting the patient. A bolus may be administered via an infusion set which also delivers basal medicine, or may be injected separately. In mental health, the bolus may be an extra dose of medicine in advance of a planned therapy session.
The basal medicine level is the amount of medicine referred to as necessary to address the patient's need for medicine absent external events, such as a diabetic patient's need for insulin between meals or other foreseen or unforeseen events, such as mealtime or snacks. The basal medicine dosage is often a lower level, delivered more regularly or nearly continuously, between external events, such as mealtimes for a diabetic patient on insulin therapy. In mental health, the basal level may be a baseline, slow acting medicine delivered via infusion, rather than through pill form, between (and through) events, such as therapy sessions.
Mood intensity might be described as how powerfully a mood is felt, and may be quantified by the likelihood or imminence of action to be taken as a direct result of mood.

As shown in the included figures, the illustrations depict instances of facial expression and changes in facial expression evaluation. The illustrations also depict instances of soliciting and receiving user input about mood and intensity. The illustrations also depict instances of reacting to the measured and input information for the user to detect and receive. The illustrations depict instances of reacting to the measured and input information for others the user has previously selected to detect and receive. However it will be understood that the invention may also be utilized for measurement of other inputs, such as audible sounds, velocity or acceleration, location, status of being indoors, outdoors, in a vehicle or other transport, altitude, body position, head position, head movement, hair length or appearance (color, styling, etc.), facial hair length or appearance, evidence of tears or other indications of crying, or injury detection, if in the future methods for such measurements are created. The invention may receive information from other sensors capable of measuring biological information, such as glucose levels, salinity, red or white blood cells, T-cell counts, dissolved oxygen, ketones, lactate, or the like on a continuous or intermittent basis, whether for information, entertainment, or compliance purposes only, as part of a feedback loop in medicine delivery, or to aid in a combination of manual and automated administration of medicine.

The included figures indicate graphical representations of mood tracked through time, such as positive and negative on an X-Y plot, with time as the independent variable X and Y as positive or negative mood. The slope of such a plot, which is the change in mood through time, say from a +4 to a +8 over the course of two hours might have a velocity expressed as (8−4)/2=4/2=2 in the positive direction. Such a mood velocity may be graphically displayed on the graph itself, for example with a different colored triangle showing a selected mood value over a selected time, forming a triangle or other geometric shape fitting the curve. Another way to display the calculated mood velocity would be a separate up or down arrow, including, but not limited to utilizing color, length, thickness, brightness, etc. typical of controls and instrumentation displays.

It would be possible to also plot the mood velocity through time, potentially as its own X-Y plot. It would be possible to similarly calculate the slope of the velocity curve, say decreasing from +2 to −2 over the course of 4 hours. In this example, that would equate to (−2−2)/4=−1 or 1 in the negative direction. Such a mood acceleration may also be displayed as a geometric figure, such as a triangle on a mood velocity plot, or as an up or down or diagonal arrow separately or could also be plotted on a separate or the same X-Y plot over time.

The mood, mood velocity, and mood acceleration could be valuable information in relation to the threshold for the app to take actions described in relation to the user or in relation to others. For example, if the threshold for taking action were at a numeric value, for example set by the user, the user's primary care physician, parent, guardian, or mental care physician at −8, if it were presumed by one or more of them that at a mood value of −10, the user may be in danger of negative behavior, such as self-harm, mood velocity and mood acceleration both provide actionable information for the user and others.

Mood intensity may be inferred by observations instantaneous or through time that taken together with the other mood measurements, indicate whether or the likelihood that action may imminently be taken as a direct result of the moods being experienced. Examples might include, but not be limited to baring of teeth, narrowing or widening of the eyes, dilation of the pupils, flush in color of the cheeks, spoken epithets or shouting or audible crying, and so on, and other data the hardware and/or software may be able to detect, such as physical change in position, velocity, or acceleration.

As a comparison, if the threshold might be imagined as a safe stopping distance from a cliff, it is worthwhile to not only know the distance of the car is 150 feet away, but also that it is still travelling at 20 miles per hour toward the cliff, but is in the process of decelerating. By contrast, if the same vehicle is 150 feet away, but travelling at 45 miles per hour toward the cliff and accelerating, the need for action for the driver to step on the brakes or the passenger in the vehicle to shout for the driver's attention is more urgent. The difference in mood intensity, by contrast, might be the difference between whether the vehicle approaching the cliff is going to push the accelerator or brakes gently, or push either one as hard as possible.

Examples of situations that could cause negative mood velocity might include, but not be limited to interactions with social media, alcohol or drugs, relationship changes, money or career stresses, death of a loved one, and so on. Examples of negative mood acceleration might be expected when these compound upon one another, such as when personal trauma leads to excessive drinking, which leads to a DUI, which could be expensive, embarrassing, and lead to loss of job or other negative consequences.

Note that it will not be necessary for the hardware and software to necessarily understand the root causes of mood, mood velocity, and mood acceleration leading to them displaying the information or triggering actions. Ideally, it will be the role of the persons or institutions to whom the user reaches out, or that initiate contact with the user as a result of the information displayed and/or shared to properly understand, assess, and act upon the information.

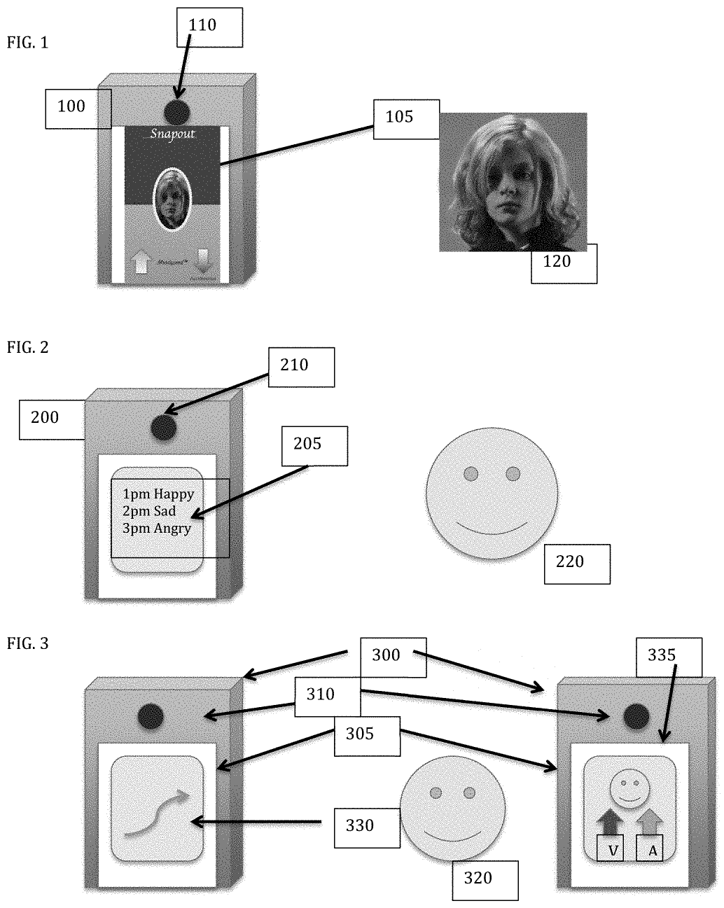

FIG. 1 shows an exemplary embodiment of application program (app) 105 on device 100, and user-facing camera 110 observing user facial expression and changes in facial expression 120 and recognizing and measuring mood and changes in mood.

FIG. 2 shows an exemplary embodiment of app 205 on screen device 200 with user-facing camera 210 observing facial expression and changes in facial expression 220 and recognizing and measuring mood and changes in mood and tracking it through time.

FIG. 3 shows an exemplary embodiment of app 305 on a device 300 with user-facing camera 310 observing facial expression and changes in facial expression 320 and recognizing and measuring mood and changes in mood and displaying mood, including graphical depiction of mood status through time 330 and graphical depiction of mood status, mood velocity, and mood acceleration 335.

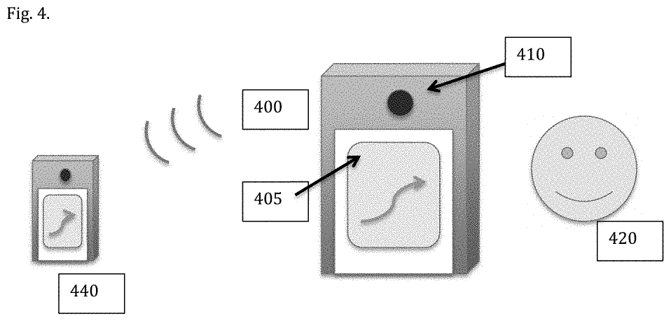

FIG. 4 shows an exemplary embodiment of app 405 on device 400 with user-facing camera 410 observing facial expression and changes in facial expression 420 and recognizing and measuring mood and changes in mood and communicating mood status and/or graphical depiction of mood status through time to others wirelessly 440.

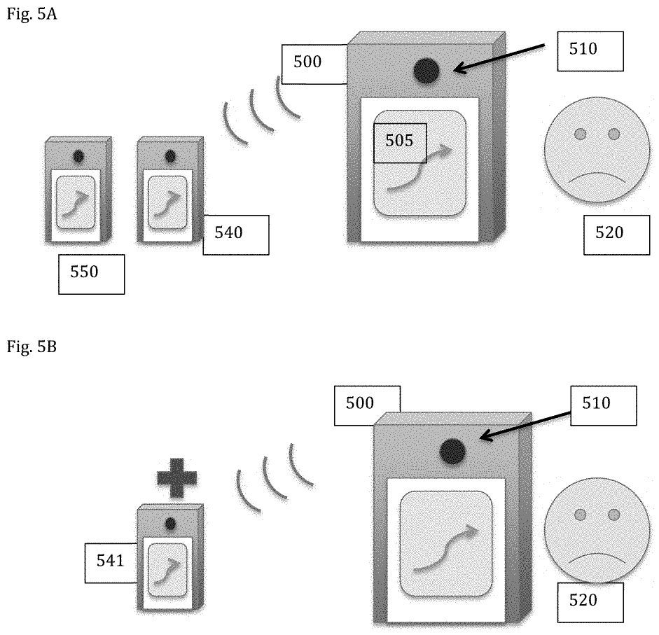

FIG. 5A shows an exemplary embodiment of app 505 on a smart phone 500 with user-facing camera 510 observing facial expression and changes in facial expression 520 and recognizing and measuring mood and changes in mood and communicating mood status and/or graphical depiction of mood status through time to others wirelessly 540 who have been pre-selected by the user and have accepted the user's invitation to share mood information.

FIG. 5B shows an exemplary embodiment of app 505 on a wearable 501 with user-facing camera 510 observing facial expression and changes in facial expression 520 and recognizing and measuring mood and changes in mood and communicating mood status and/or graphical depiction of mood status through time to others wirelessly 541 who are health care professionals or first responder professionals.

FIG. 5C shows an exemplary embodiment of app 505 on a tablet 502 with user-facing camera 510 observing facial expression and changes in facial expression 520 and recognizing and measuring mood and changes in mood and communicating mood status and/or graphical depiction of mood status through time to others wirelessly 542 who are health care institutions or first responder institutions wirelessly.

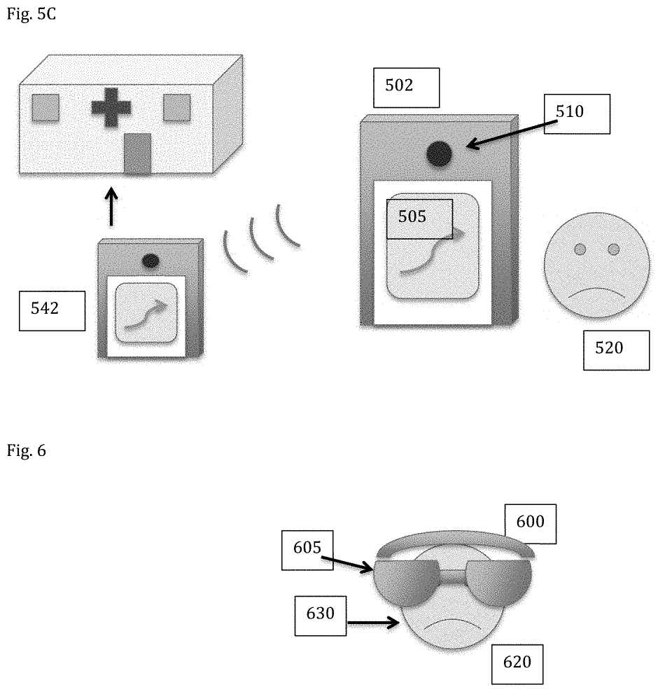

FIG. 6 shows an exemplary embodiment of app 605 on VR interface 600 with the app capable of querying or receiving user input 630 about mood simultaneous with facial recognition hardware and software evaluating user expression and changes in facial expression to improve mood determination algorithms.

FIG. 7 shows an exemplary embodiment of app 705 on device 700 with the app capable of querying or receiving user input 730 about mood simultaneous with facial recognition hardware and software evaluating user expression and changes in facial expression to determine mood and changes in mood.

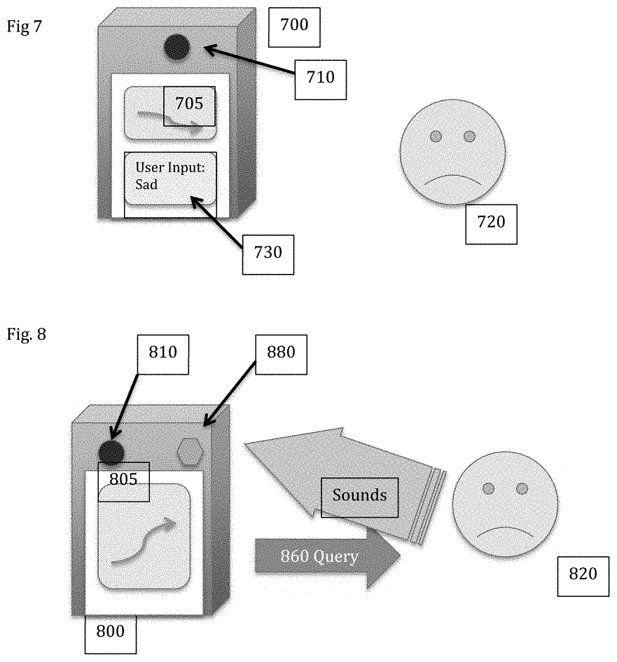

FIG. 8 shows an exemplary embodiment of app 805 on device 800 with the app capable of 860 querying and/or receiving sounds through a microphone 880 simultaneous with facial recognition hardware and software evaluating user expression and changes in facial expression to determine mood and changes in mood.

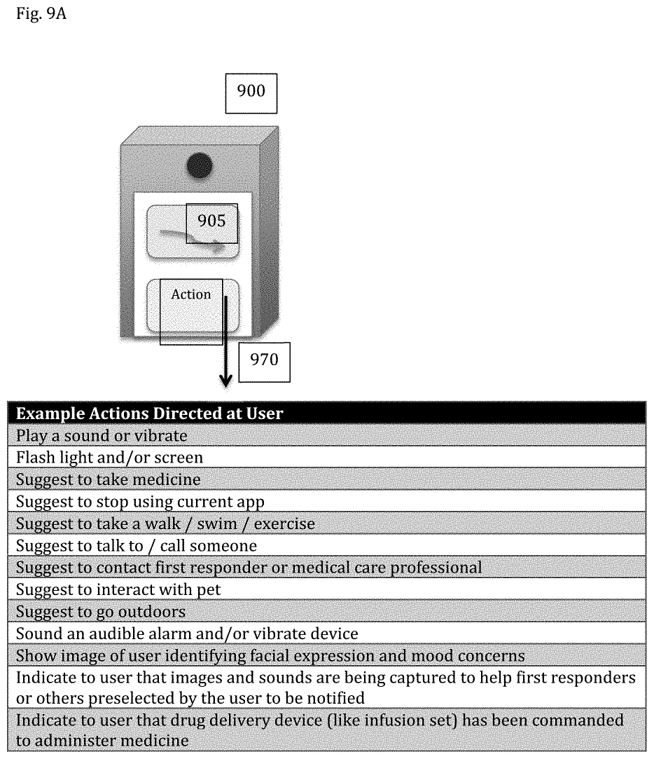

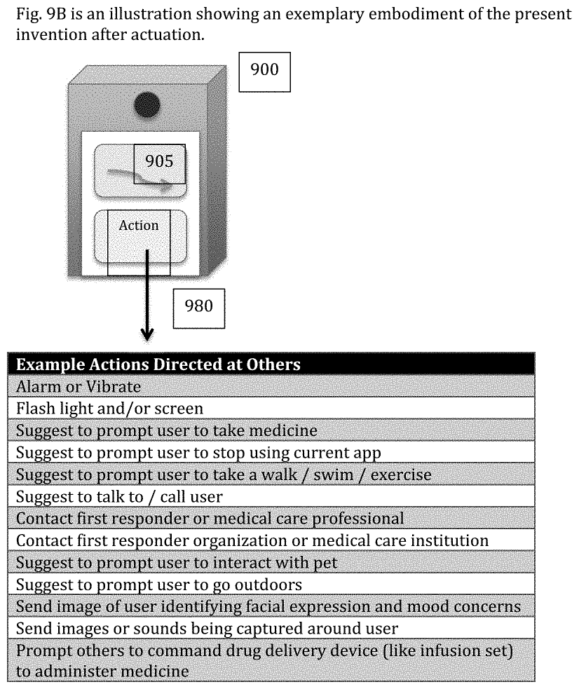

FIG. 9A shows an exemplary embodiment of app 905 on device 900 with the app capable of initiating actions 970 directed at the user, preset by the user, and based upon mood determination.

FIG. 9B shows an exemplary embodiment of app 905 on device 900 with the app capable of initiating actions 980 directed at others, preset by the user, and based upon mood determination.

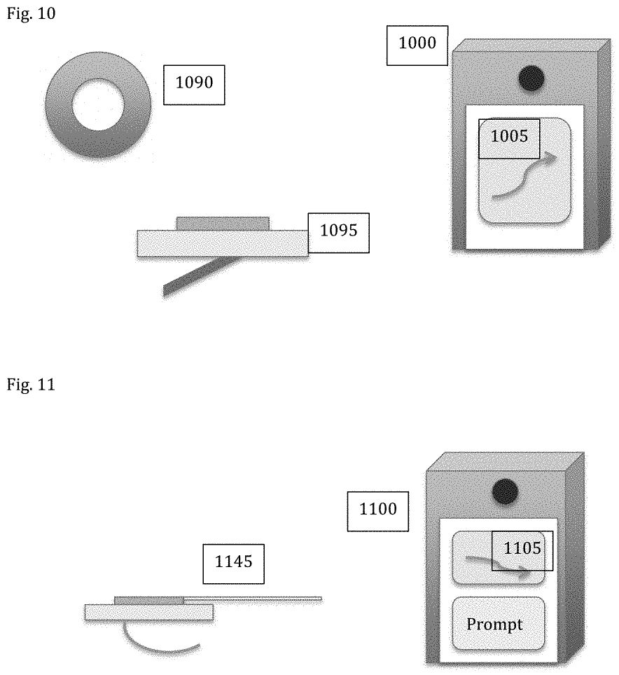

FIG. 10 shows an exemplary embodiment of an app 1005 on device 1000 with the app capable of querying or receiving information inputs from wearable 1090 and/or biological sensor 1095 simultaneous with facial recognition hardware and software evaluating user expression and changes in facial expression to determine mood and changes in mood.

FIG. 11 shows an exemplary embodiment of an app 1105 on device 1100 with the app capable of prompting the user to manually interact with a medicine delivery device, such as an infusion set 1145, to deliver or adjust a continuous (basal), intermittent, or one-time (bolus) dose of medicine based upon facial recognition hardware and software evaluating user expression and changes in facial expression to determine mood and changes in mood.

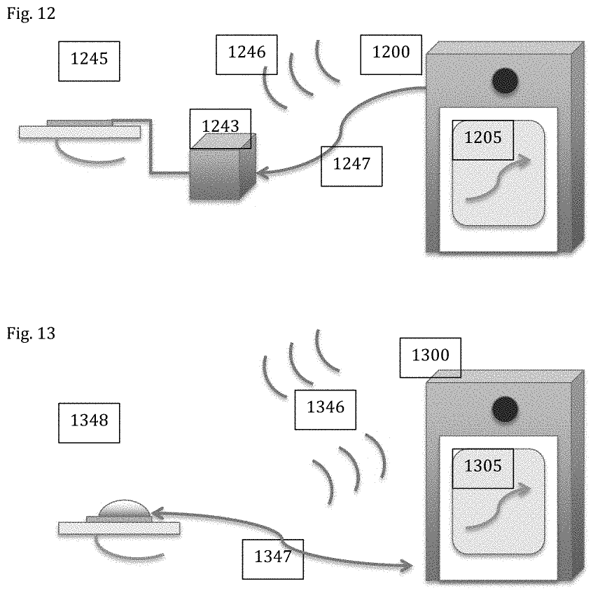

FIG. 12 shows an exemplary embodiment of an app 1205 on device 1200 with the app capable of interacting with a medicine delivery device, such as a medicine pump 1243 with an infusion set 1245 wirelessly 1246 or through wired communication signal 1247, to deliver or adjust a continuous (basal), intermittent, or one-time (bolus) dose of medicine based upon facial recognition hardware and software evaluating user expression and changes in facial expression to determine mood and changes in mood.

FIG. 13 shows an exemplary embodiment of an app 1305 on device 1300 with the app capable of interacting with a medicine delivery device, such as a patch pump infusion set 1348 wirelessly 1346 or through wired communication signal 1347, in a feedback loop, to automatically (without manual user input) deliver or adjust a continuous (basal), intermittent, or one-time (bolus) dose of medicine based upon facial recognition hardware and software evaluating user expression and changes in facial expression to determine mood and changes in mood and receive information about medicine status, such as flow, pressure, absorption rate, etc.

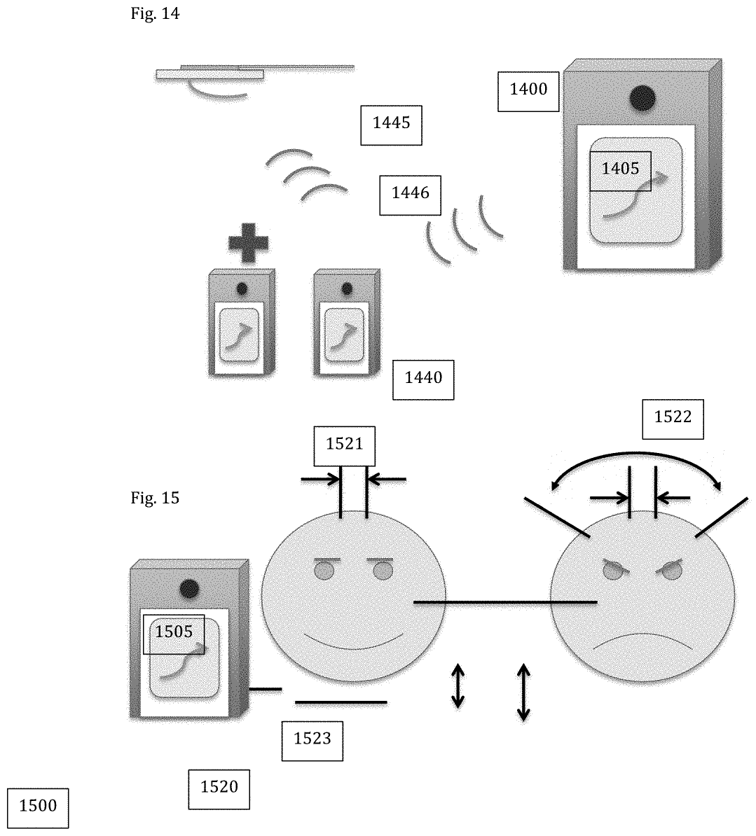

FIG. 14 shows an exemplary embodiment of an app 1405 on device 1400 with the app capable of interacting with a medicine delivery device, such as an infusion set 1445, to allow others (such as family members, first responders, or healthcare professionals with prior consent of user or user's parents or guardians) 1440 to wirelessly (without manual user input) 1446 deliver or adjust a continuous (basal), intermittent, or one-time (bolus) dose of medicine based upon facial recognition hardware and software evaluating user expression and changes in facial expression to determine mood and changes in mood.

FIG. 15 shows an exemplary embodiment of an app 1505 on a device 1500 with user-facing camera 1510 observing facial expression and changes in facial expression 1520 and recognizing and measuring mood and changes in mood and displaying mood, including measurement of biometric data including, but not limited to: distance between eyebrows 1521, angle of eyebrows 1522, distance from corners of mouth to bottom of eyes 1523, etc. This list is illustrative, and not exhaustive.

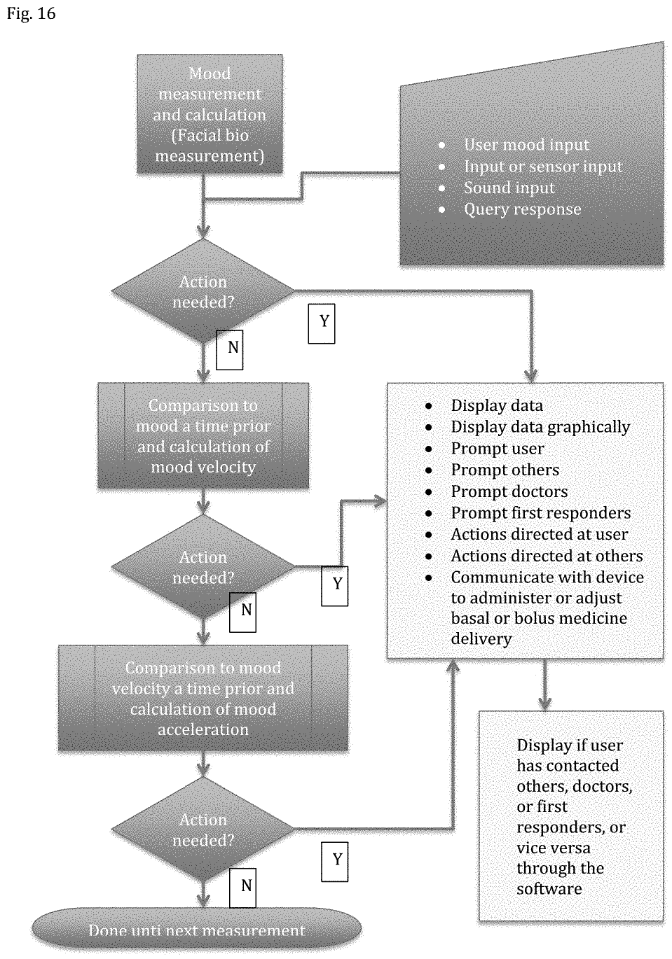

FIG. 16 show an exemplary embodiment of an application on a device with a flowchart for facial recognition hardware and software to measure and calculate mood from direct measurement or alternate inputs, such as user input, sounds, or user response to query from the product. The flowchart demonstrates utilizing instantaneous information and information through time to measure and calculate mood, mood velocity, and mood acceleration to compare to reference levels to determine if action is necessary, and take that action in the event it is necessary. It also shows an example of those using tracking software of being able to observe if the user has contacted others through the app and tracking app or vice versa, for example to reduce concerns if that person is unreachable due to interaction with others to address the mood.

Common and familiar methods and assemblies may not be mentioned, in order to be brief and clear.

While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims.

What is claimed, is:

1. An application for an electronic computing device, comprising the steps of:
capturing a first facial image of a person at a first time;
capturing a second facial image of a person at a second time;
evaluating a change in the first and second facial images;
determining, using the evaluated change, a mood characteristic; and
taking an action based on the determined mood characteristic.

2. The application according to claim 1, wherein the mood characteristic is an instantaneous mood of the first facial image or the second facial image.

3. The application according to claim 1, wherein the mood characteristic is an acceleration or the velocity of the mood change between the first facial image and the second facial image.

4. The application according to claim 1, wherein the mood characteristic is an intensity of the mood of the first facial image or the second facial image.

5. The application according to claim 1, wherein the action is using a display on the electronic device to present an indicia reflecting the mood characteristic.

6. The application according to claim 1, wherein the electronic device has a wireless radio, and the action is communicating a message indicative of the mood characteristic to a third party.

7. The application according to claim 1, wherein the electronic device has a wireless radio, and the action is communicating a message to emergency or health care personnel to seek help for the person.

8. The application according to claim 1, wherein the action is to request, using the electronic device, information from the person with regard to the mood characteristic.

9. The application according to claim 1, wherein the action is to present a visual alarm, a sound alarm, or a vibration, to the person.

10. The application according to claim 1, wherein the action is to present the user with information regarding a proper dosage of a medication to self-administer.

11. The application according to claim 1, wherein the action is to automatically administer a dose of medication to the person using an infusion set.

12. The application according to claim 1, further including sensors to collect biometric data, and using the biometric data in determining the mood characteristic.

13. The application according to claim 12, wherein the biometric data is pulse, temperature, sound level, tone of voice, color of skin, presence of moisture, such as sweat, tears, or blood, or location.

14. The application according to claim 1, wherein the mood characteristic indicates a possible imminent suicide risk for the person.

15. An application for an electronic computing device, comprising the steps of:
receiving and storing data reflecting a target mood;
capturing a facial image of a person;
evaluating a change between the target mood and the captured mood;
determining, using the evaluated change, a mood characteristic; and
taking an action based on the determined mood characteristic.

16. The application according to claim 15, wherein the mood characteristic is an instantaneous mood of the facial image, or a mood change between the target mood and the mood of the captured image.

17. The application according to claim 15, wherein the electronic device has a wireless radio, and the action is communication a message indicative of the mood characteristic to a third party.

18. The application according to claim 15, wherein the electronic device has a wireless radio, and the action is communication a message to emergency or health care personnel to seek help for the person.

19. The application according to claim 15, wherein the mood characteristic indicates a possible imminent suicide risk for the person.

20. The application according to claim 15, further including sensors to collect biometric data, and using the biometric data in determining the mood characteristic, the biometric data being pulse, temperature, sound level, tone of voice, color of skin, presence of moisture, such as sweat, tears, or blood, or location.
